(19) United States
(12) Patent Application Publication
(10) Pub. No.: US 2025/0122457 A1
(43) Pub. Date: Apr. 17, 2025

(54) CULTURING DEVICE

(71) Applicant: NATIONAL KAOHSIUNG UNIVERSITY OF SCIENCE AND TECHNOLOGY, Kaohsiung City (TW)

(72) Inventors: Chi-Chang HSIEH, Kaohsiung City (TW); Tsung-Ming CHEN, Kaohsiung City (TW); Yen-Yi LI, Kaohsiung City (TW); Min-Hong CHEN, Kaohsiung City (TW)

(73) Assignee: NATIONAL KAOHSIUNG UNIVERSITY OF SCIENCE AND TECHNOLOGY, Kaohsiung City (TW)

(21) Appl. No.: 18/540,442

(22) Filed: Dec. 14, 2023

(30) Foreign Application Priority Data
Oct. 13, 2023 (TW) 112139179

(51) Int. Cl. C12M 1/00

(52) U.S. Cl. CPC C12M 23/38; C12M 23/22; C12M 31/10

(57) ABSTRACT

A culturing device includes a culturing container, a transparent plate, a cover and a light emitting assembly. The culturing container includes a body portion and an opening portion. The body portion is connected to the opening portion. The opening portion has an opening. The transparent plate is disposed on the opening portion, and covers the opening. The cover is disposed on a side of the transparent plate located farthest away from the culturing container. The cover and the transparent plate together form an airflow channel therebetween. The light emitting assembly is disposed on the cover, and is located in the airflow channel.

### CROSS-REFERENCE TO RELATED APPLICATIONS

This non-provisional application claims priority under 35 U.S.C. § 119 (a) on Patent Application No(s). 112139179 filed in ROC, on Oct. 13, 2023, the entire contents of which are hereby incorporated by reference.

### TECHNICAL FIELD

The present disclosure relates to a culturing device, more particularly to a culturing device having an airflow channel.

### BACKGROUND

The growth of organisms is greatly affected by the growth environment. Conventionally, certain organisms can only be cultured in specific seasons. With the advancement of technology and a better understanding of the growth environment of the organisms, the organisms can be cultured in arbitrary season via controlling the growth environment.

Generally, culturing lights with different colors can be installed on the culturing device to emit lights to the culturing organisms so as to promote the growth of different culturing organisms with the lights with suitable color. Since the culturing lights generate heat when emitting lights, a cooling device is required to be additionally disposed on the culturing device to cool the culturing lights so as to keep the normal operation of the culturing lights. However, additional cooling device causes a volume of the culturing device to be large, such that a space occupied by the culturing device is increased, thereby causing the inconvenience of culturing. Therefore, how to prevent the volume of the culturing device from being increased to reduce the space occupied by the culturing device is an important issue to be solved.

### SUMMARY

The present disclosure provides a culturing device which can effectively cool a light emitting assembly without increasing the volume thereof.

One embodiment of the disclosure provides a culturing device including a culturing container, a transparent plate, a cover and a light emitting assembly. The culturing container includes a body portion and an opening portion. The body portion is connected to the opening portion. The opening portion has an opening. The transparent plate is disposed on the opening portion, and covers the opening. The cover is disposed on a side of the transparent plate located farthest away from the culturing container. The cover and the transparent plate together form an airflow channel therebetween. The light emitting assembly is disposed on the cover, and is located in the airflow channel.

According to the culturing device as described in the above embodiments, the transparent plate and the cover of the culturing device together form the airflow channel therebetween, such that the airflow can flow through the airflow channel to cool the light emitting assembly located in the airflow channel without installing additional cooling assembly. Accordingly, the volume of the culturing device is prevented from being increased.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a perspective view of a culturing device in accordance with an embodiment of the disclosure;

FIG. 2 is an exploded view of the culturing device in FIG. 1;

FIG. 3 is another exploded view of the culturing device in FIG. 1;

FIG. 4 is a cross-sectional view of the culturing device in FIG. 1; and

FIG. 5 is a plane view of a light emitting assembly of the culturing device in FIG. 1.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In addition, the terms used in the present disclosure, such as technical and scientific terms, have its own meanings and can be comprehended by those skilled in the art, unless the terms are additionally defined in the present disclosure. That is, the terms used in the following paragraphs should be read on the meaning commonly used in the related fields and will not be overly explained, unless the terms have a specific meaning in the present disclosure.

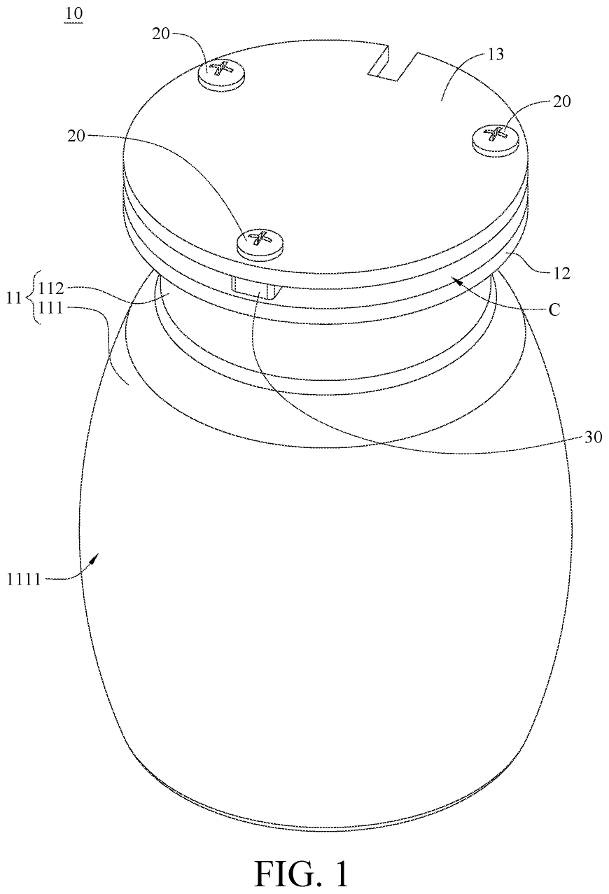

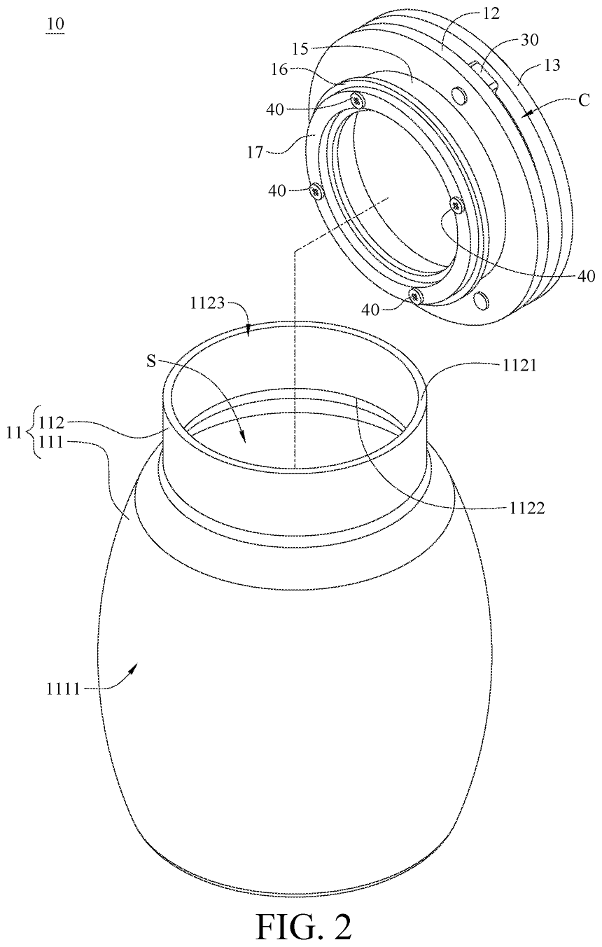

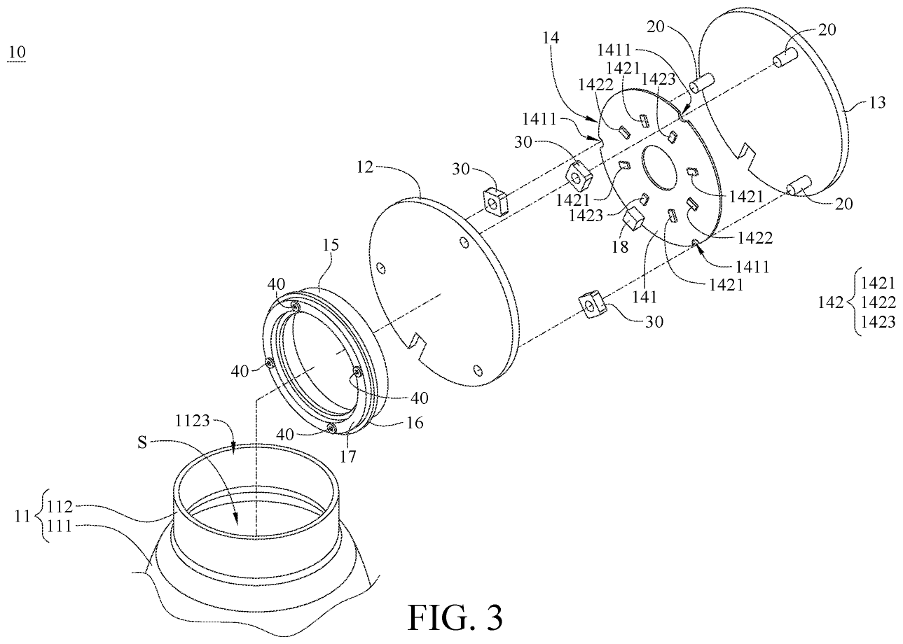

Please refer to FIG. 1 to FIG. 3, where FIG. 1 is a perspective view of a culturing device 10 in accordance with an embodiment of the disclosure, FIG. 2 is an exploded view of the culturing device 10 in FIG. 1, and FIG. 3 is another exploded view of the culturing device 10 in FIG. 1.

In this embodiment, the culturing device 10 includes a culturing container 11, a transparent plate 12, a cover 13, a light emitting assembly 14, an extension mounting member 15 and a sealing member 16. A cross-section of the culturing container 11 is, for example, circular. The culturing container 11 has a culturing space S. The culturing space S is configured to accommodate a culturing liquid (not shown) and culturing organisms (not shown). The culturing liquid is, for example, seawater. The culturing organisms are, for example, corals.

The culturing container 11 includes a body portion 111 and an opening portion 112. The body portion 111 is connected to the opening portion 112. The body portion 111 has a curved surface 1111. The curved surface 1111 extends from a side of the body portion 111 located closest to the opening portion 112 to a side of the body portion 111 located farthest away from the opening portion 112. Also, a diameter of the body portion 111 of the culturing container 11 decreases gradually from a central section of the body portion 111 toward two opposite sides of the body portion 111. The opening portion 112 includes a top side 1121 and a bottom side 1122 that are opposite to each other. The opening portion 112 has an opening 1123.

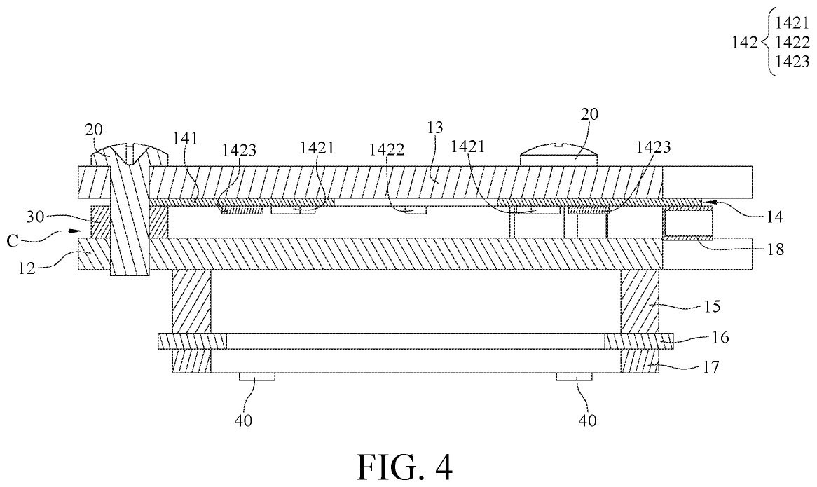

Please refer to FIG. 1 to FIG. 4, where FIG. 4 is a cross-sectional view of the culturing device 10 in FIG. 1. The transparent plate 12 is disposed on the top side 1121 of the opening portion 112, and covers the opening 1123. The cover 13 is disposed on a side of the transparent plate 12 located farthest away from the culturing container 11. The cover 13 and the transparent plate 12 together form an airflow channel C.

Specifically, the culturing device 10 further includes a plurality of first fasteners 20 and a plurality of spacers 30. The spacers 30 are disposed at the first fasteners 20. The first fasteners 20 are, for example, screws, and the spacers 30 are, for example, standoffs. The transparent plate 12 is fixed to the cover 13 via the first fasteners 20, and is spaced apart from the cover 13 via the spacers 30 so as to form an airflow channel C therebetween. The light emitting assembly 14 is located in the airflow channel C, such that an airflow (not shown) can cool the light emitting assembly 14 via flowing through the airflow channel C.

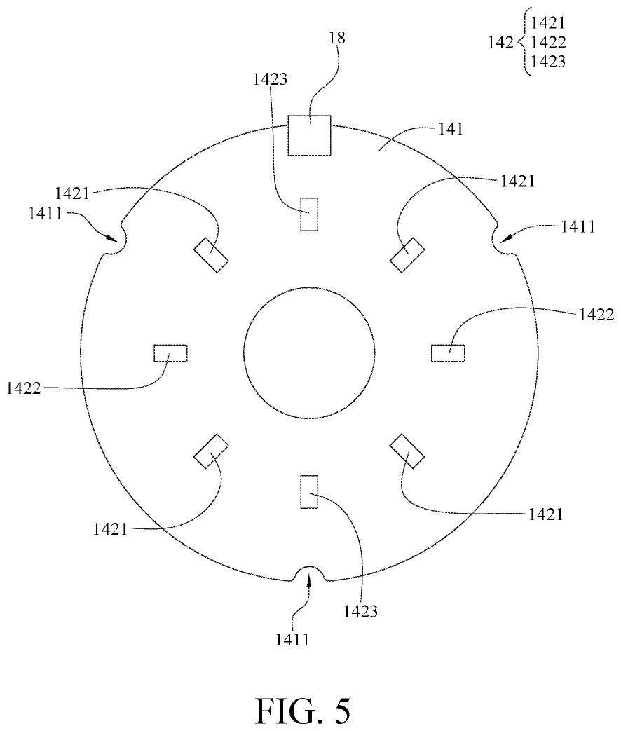

Please refer to FIG. 1 to FIG. 5, where FIG. 5 is a plane view of a light emitting assembly 14 of the culturing device 10 in FIG. 1. The light emitting assembly 14 includes a circuit board 141 and a plurality of light emitting components 142. The circuit board 141 has a plurality of positioning recesses 1411. The first fasteners 20 are located in the positioning recesses 1411, respectively, such that the circuit board 141 can be positioned on the cover 13.

The light emitting components 142 are disposed on the circuit board 141, and include a plurality of white light emitting diodes 1421, a plurality of blue light emitting diodes 1422 and a plurality of ultraviolet light emitting diodes 1423. The white light emitting diodes 1421, the blue light emitting diodes 1422 and the ultraviolet light emitting diodes 1423 are selectively configured to emit lights according to growth conditions of the culturing organisms. The lights emitted from the white light emitting diodes 1421, the blue light emitting diodes 1422 and the ultraviolet light emitting diodes 1423 are transferred toward the culturing organisms located in the culturing space S through the transparent plate 12.

Two opposite sides of the extension mounting member 15 are connected to the transparent plate 12 and the sealing member 16, respectively. Specifically, the culturing device 10 further includes a plurality of second fasteners 40 and a positioning ring 17. The second fasteners 40 are, for example, screws. The sealing member 16 is sleeved on the positioning ring 17. A side of the extension mounting member 15 is connected to the transparent plate 12 via, for example, an adhesive (not shown), and the other side of the extension mounting member 15 is fixed to the sealing member 16 and the positioning ring 17 via the second fasteners 40.

The opening portion 112 surrounds the extension mounting member 15. The sealing member 16 is disposed on the bottom side 1122 of the opening portion 112. A diameter of the sealing member 16 is, for example, less than a diameter of the transparent plate 12, and the diameter of the sealing member 16 is, for example, larger than a diameter of the opening portion 112. Accordingly, a sealing effect of the opening portion 112 can be improved. Therefore, a gas located in the culturing space S can be prevented from being mixed with an air located out of the culturing space S via the opening portion 112 to prevent a culturing environment for the culturing organisms from being changed.

In this embodiment, the transparent plate 12 and the cover 13 of the culturing device 10 together form the airflow channel C therebetween, such that the airflow can flow through the airflow channel C to cool the light emitting assembly 14 located in the airflow channel C without installing additional cooling assembly. Accordingly, the volume of the culturing device 10 is prevented from being increased.

In addition, the culturing device 10 includes the extension mounting member 15 and the sealing member 16. The sealing member 16 is located on the bottom side 1122 of the opening portion 112 via the extension mounting member 15 to seal the opening 1123 of the opening portion 112. Accordingly, the gas located in the culturing space S can be prevented from being mixed with the air located out of the culturing space S. Therefore, the culturing environment for the culturing organisms of culturing device 10 can be maintained.

Moreover, the cross-section of the culturing container 11 is circular, and the body portion 111 of the culturing container 11 has a curved surface 1111. Accordingly, after the white light emitting diodes 1421, the blue light emitting diodes 1422 and the ultraviolet light emitting diodes 1423 emit lights to the culturing space S of the culturing container 11, most of the emitted lights are reflected within the culturing space S of the culturing container 11 and are prevented from being escaped from the culturing space S. Therefore, the culturing environment for the culturing organisms can be maintained.

In this embodiment, the culturing device 10 further includes a connector 18. The connector 18 is disposed on the circuit board 141, and is configured to be electrically connected to an external cable (not shown) and the light emitting components 142. Accordingly, the external cable can transmit external power to the light emitting components 142 via the connector 18, such that the light emitting components 142 can emit light after receiving the external power.

In this embodiment, the white light emitting diodes 1421, the blue light emitting diodes 1422 and the ultraviolet light emitting diodes 1423 of the light emitting components 142 are arranged as shown in FIG. 5, but the disclosure is not limited thereto. In other embodiments, the white light emitting diodes, the blue light emitting diodes and the ultraviolet light emitting diodes of the light emitting components may be arranged in other ways.

In this embodiment, the side of the extension mounting member 15 is connected to the transparent plate 12 via the adhesive, but the disclosure is not limited thereto. In other embodiments, the extension mounting member and the transparent plate may be, for example, integrally formed as a single piece.

In this embodiment, the light emitting assembly 14 is cooled by one or more fans external to the culturing device 10, but the disclosure is not limited thereto. In other embodiments, the culturing device may include one or more fans to cool the light emitting assembly.

In this embodiment, the culturing device 10 further includes a connector 18. The connector 18 is configured to be electrically connected to an external cable and the light emitting components 142, such that the light emitting components 142 can receive the external power to emit light via the connector 18 electrically connected to the external cable. However, the disclosure is not limited thereto. In other embodiments, the culturing device may include a power storage unit, and does not include the connector. The power storage unit is, for example, a battery. The light emitting components is electrically connected to the power storage unit, such that the light emitting components can receive a power provided by the power storage unit to emit lights.

According to the culturing device as described in the above embodiments, the transparent plate and the cover of the culturing device together form the airflow channel therebetween, such that the airflow can flow through the airflow channel to cool the light emitting assembly located in the airflow channel without installing additional cooling assembly. Accordingly, the volume of the culturing device is prevented from being increased.

In addition, the culturing device includes the extension mounting member and the sealing member. The sealing member is located on the bottom side of the opening portion via the extension mounting member to seal the opening of the opening portion. Accordingly, the gas located in the culturing space can be prevented from being mixed with the air located out of the culturing space. Therefore, the culturing environment for the culturing organisms of culturing device can be maintained.

It will be apparent to those skilled in the art that various modifications and variations can be made to the present disclosure. It is intended that the specification and examples be considered as exemplary embodiments only, with a scope of the disclosure being indicated by the following claims.

What is claimed is:

1. A culturing device, comprising:
a culturing container, comprising a body portion and an opening portion, wherein the body portion is connected to the opening portion, and the opening portion has an opening;
a transparent plate, disposed on the opening portion and covering the opening;
a cover, disposed on a side of the transparent plate located farthest away from the culturing container, wherein the cover and the transparent plate together form an airflow channel therebetween; and
a light emitting assembly, disposed on the cover and located in the airflow channel.

2. The culturing device according to claim 1, further comprising an extension mounting member and a sealing member, wherein two opposite sides of the extension mounting member are connected to the transparent plate and the sealing member, respectively, the opening portion surrounds the extension mounting member, the opening portion comprises a top side and a bottom side that are opposite to each other, the transparent plate is disposed on the top side, and the sealing member is disposed on the bottom side.

3. The culturing device according to claim 2, wherein the extension mounting member and the sealing member are integrally formed as a single piece.

4. The culturing device according to claim 2, wherein a diameter of the sealing member is less than a diameter of the transparent plate, and the diameter of the sealing member is larger than a diameter of the opening portion.

5. The culturing device according to claim 1, wherein the light emitting assembly comprises a circuit board and at least one light emitting component, the circuit board is disposed on the cover, and the at least one light emitting component is disposed on the circuit board.

6. The culturing device according to claim 5, further comprising a connector, wherein the connector is disposed on the circuit board, and the connector is electrically connected to the at least one light emitting component.

7. The culturing device according to claim 5, wherein the light emitting assembly comprises a plurality of light emitting components, and the plurality of light emitting components comprise at least one white light emitting diode and at least one blue light emitting diode.

8. The culturing device according to claim 7, wherein the plurality of light emitting components further comprise at least one ultraviolet light emitting diode.

9. The culturing device according to claim 1, wherein the body portion of the culturing container has a curved surface, the curved surface extends from a side of the body portion located closest to the opening portion toward a side of the body portion located farthest away from the opening portion.

10. The culturing device according to claim 1, wherein a cross-section of the culturing container is circular.
